(19) United States
(12) Patent Application Publication
(10) Pub. No.: US 2007/0105215 A1
(43) Pub. Date: May 10, 2007

(54) METHOD OF CULTURING STEM CELLS USING ABC TRANSPORTERS

(75) Inventors: Young Sun LEE, Seongnam-si, Gyeonggi-do (KR); Byung Chul KIM, Suwon-si, Gyeonggi-do (KR); Ok Ryul SONG, Seoul (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD., Suwon-si (KR)

(21) Appl. No.: 11/556,392

(22) Filed: Nov. 3, 2006

(30) Foreign Application Priority Data
Nov. 3, 2005 (KR) 10-2005-0104943

(51) Int. Cl. C12N 5/06; C12N 5/08

(52) U.S. Cl. 435/325; 435/366

(57) ABSTRACT

Disclosed herein is a method of culturing stem cells, which can remove differentiated cells generated in stem cell culture, using ABC transporters, according to a stem cell's characteristic in that the stem cells contain a plurality of the ABC transporters. The method comprises the steps of: bringing a stem cell culture into contact with an antitumor drug or toxin as a substrate of ATP-Binding Cassette transporters (ABC transporters) to allow the substrate to react with the stem cell culture; and reculturing viable cells among the stem cell culture which reacted with the substrate, the viable cells having no substrate introduced therein. According to the disclosed method, the differentiated cells generated in stem cell culture can be easily removed using an antitumor drug or toxin among various substrates of the ABC transporters. Thus, the method has an excellent effect capable of significantly increasing the purity of a stem cell culture.

This application claims priority to Korean Patent Application No. 10-2005-0104943, filed Nov. 3, 2005, and all the benefits accruing therefrom under 35 U.S.C. § 119, the entire contents of which are hereby incorporated by reference.

### BACKGROUND OF THE INVENTION

1. Field of the Invention

The present invention relates to a method of culturing stem cells, and more particularly to a method of culturing Side population (SP) stem cells, which can remove differentiated cells generated in stem cell culture, using ABC transporters, according to a stem cell's characteristic in that the stem cells contain a plurality of the ABC transporters.

2. Description of the Prior Art

The term “stem cell” refers to an undifferentiated cell that has the ability to divide and replicate itself for an indefinite period while maintaining an undifferentiated state even ex vivo. Also, the stem cell has the ability to differentiate into many kinds of different cells in an organism depending on the developmental stage and location of an individual. When this stem cell is extracted and transplanted into patient's lesions, injured cells can be restored to the original state, so that diseases can be treated or tissues and organs can be regenerated. Also, researchers state that a method capable of preventing congenital genetic diseases can be found through deep studies on embryonic stem cells.

Human stem cells can be classified into three categories: totipotent cells which are formed in the initial division stage of embryos (up to the 8-cell stage after the fertilization of eggs with sperms); embryonic stem cells (pluripotent cells) which are formed by the continued division of the totipotent cells; and multipotent cells (adult stem cells) which are found in mature tissues and organs.

However, the totipotent cell formed in the initial division stage can itself become an embryo, studies thereon have been limited for an ethical reason. Thus, interest in current studies is concentrated on pluripotent cells and multipotent cells.

As used with respect to embryonic stem cells, the term “embryo” refers to a fertilized embryo produced by the fusion of an egg with a sperm. Usually, it refers to a developing organism from implantation until the eighth week of pregnancy, at which differentiation into tissues and organs is completed. The term “embryonic stem cell” refers to a stem cell extracted from either a blastocyst-stage embryo just before conception or a fetus miscarried between the eighth to twelfth weeks of pregnancy, and can differentiate into all cells forming the human body. Regarding this, the term “embryonic stem cell line” refers to a state in which a primitive cell having the ability to develop into various organs is maintained by inhibiting the differentiation thereof.

In comparison with the embryonic stem cells, adult stem cells are produced by extracting cells already present in various human organs and developing the extracted cells into stem cells, and are characterized in that they differentiate only into a specific tissue. Furthermore, the adult stem cells have problems in that they have short lifespan and are extracted in small amounts, compared to the embryonic stem cells, and thus are difficult to use in practical applications. However, the adult stem cells receive attention, because experiments of differentiating the adult stem cells into various tissues such as liver cells were recently successful.

The hematopoietic stem cells is major adult stem cell. A promising and increasingly exploited property of hematopoietic stem cells is their ability to efflux the fluorescent dye Hoechst 33342. The Hoechst-negative cells are isolated by fluorescence activated cell sorting as a so-called side “population” (SP) of bone marrow. This SP from bone marrow, as well as other tissues, is reported to contain immature stem cells with considerable plasticity. Some cell lines also efflux Hoechst and generate SP profiles.

Hence, the isolated cells are termed side-population (SP) cells. This low-staining SP is lost after treatment with verapamil, which has led to the assumption that the MDR1-encoded adenosine triphosphate-binding cassette (ABC) transporter, P-glycoprotein (P-gp), is responsible for Hoechst dye efflux in these cells. Recently, excitement has been generated by the finding that putative stem cells from solid tissues may also share this SP phenotype.

However, in a process of culturing adult stem cells, a phenomenon preventing the expansion of stem cells occurs, because differentiated cells other than stem cells are naturally generated and cultured together with the stem cells. Nevertheless, a reliable mechanism for the expansion of stem cells is not yet found, and thus stem cells are substantially always cultured together with differentiated cells.

In an attempt to solve this problem, the FACS (fluorescence activated cell sorter) method performs the isolation of stem cells using a fluorescence-labeled marker binding specifically to the stem cells. This method has excellent isolation ability, but is disadvantageous in that it requires a large and expensive system. Also, the MACS (magnetic cell sorting) method is a method of isolating stem cells using a magnetic particle-labeled marker binding specifically to the stem cells. This MACS method has a shortcoming in that it is difficult to achieve quantification, whereas it has excellent isolation ability, uses an inexpensive system and is performed in a simple and rapid manner, and thus it is most frequently used.

Also, there was an attempt to expand only stem cells by optimizing medium additives such as cytokine, Cu chelate, tetraethylene pentamine (TEPA) and the like, but this method is also unsuitable, because it requires expensive substances and still shows differentiated cells.

Specifically, European Patent No. 1424388 discloses a method for rapidly expanding adult stem cells using a special culture medium composition while preventing differentiation. However, even when the culture of stem cells is performed using the technology disclosed in said patent, the stem cells still cannot be prevented from being cultured together with differentiated cells.

In an attempt to solve this problem, U.S. Pat. No. 5,674,750 discloses a technique of expanding only the desired cells (i.e., stem cells) by harvesting expanded stem cells through a column. Also, U.S. Pat. No. 5,925,567 discloses a technique comprising removing unwanted cells (i.e., differentiated cells) through a column) and expanding only the remaining stem cells.

In the techniques disclosed in said patents, there are problems in that the expanded cells cannot be completely separated into stem cells and differentiated cells, and a process for this separation is highly complicated.

### SUMMARY OF THE INVENTION

The present inventors have conducted studies on adult stem cells to solve the above-described problems occurring in the prior art and, as a result, found that, because, one of the adult stem cells, Side population (SP) stem cells have a characteristic in that their membranes contain a plurality of ATP-Binding Cassette transporters (ABC transporters) involved in the active transport of substances, stem cells among cultured cells have the ABC transporters, whereas differentiated cells have little or no ABC transporter, thereby completing the present invention. Also, in the hematopoiesis process, putative stem cells differentiate into progenitor cells, during which the expression levels of ABC transporter genes in the cells are different from each other. Specifically, in the putative stem cells (SP, CD34+/KDR+, and CD34+/CD38−), the expression level of the ABC transporter genes is high, but in the differentiated progenitor cell, the expression level is rapidly decreased (Blood. 2002; 99:507-512). In other words, the stem cells and the differentiated cells are different from each other with respect to the ABC transporters.

Accordingly, it is a main object of the present invention to a method of culturing stem cells using ABC transporters, which can increase the purity of cultured stem cells by selectively removing only differentiated cells from a stem cell culture, which contains stem cells containing a plurality of ABC transporters, and differentiated cells containing no ABC transporter, using a substrate of ABC transporters, which is an antitumor drug or toxin.

To achieve the above object the present invention provides a method of culturing stem cells using ABC transporters, the method comprising the steps of: bringing a stem cell culture into contact with a substrate of ATP-Binding Cassette transporters (ATP transporters) as an antitumor drug or toxin, to allow the stem cell culture to react with the substrate; and reculturing viable cells among the stem cell culture which reacted with the substrate, the viable cells having no substrate introduced therein.

### BRIEF DESCRIPTION OF THE DRAWINGS

The above and other objects, features and advantages of the present invention will be more clearly understood from the following detailed description taken in conjunction with the accompanying drawings, in which:

FIG. 1 schematically shows the structures of MDR1, MRP1 and ABCG2 among ABC transporters;

FIG. 2 is a schematic diagram showing the culture method according to the present invention, in which differentiated cells containing no ABC transporter have a substrate as an anti-tumor drug or toxin in the cells thereof, and stem cells having ABC transporters therein substantially have pumping out a substrate as an anti-tumor drug or toxin in the cells;

FIG. 3 is a graphic diagram showing the results of measurement conducted in Example 1 and Comparative Example 1;

FIG. 4 is a graphic diagram showing the results of measurements conducted in Example 1 and Comparative Example 1;

FIG. 5 is a graphic diagram showing the results of measurements conducted in Example 3; and

FIG. 6 is a graphic diagram showing the results of Test Examples 1 and 2.

### DETAILED DESCRIPTION OF THE INVENTION

The invention now will be described more fully herein after with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

To accomplish this, there is provided a method of culturing stem cells using ABC transporters, the method comprising the steps of: bringing a stem cell culture into contact with a substrate of ATP transporters (ATP-Binding Cassette transporters) as an antitumor drug or toxin, to allow the stem cell culture to react with the substrate; and reculturing viable cells among the stem cell culture which reacted with the substrate, the viable cells having no substrate introduced therein.

As used herein, the ATP-Binding Cassette transporters (ABC transporters) are membrane proteins, which have an ATP-binding site and actively transport substances from the cytoplasm to the outside of cells using the ATP energy. Among these ABC transporters, those frequently found in stem cells include Multi-drug resistance (MDR), Multi-drug resistance associated protein (MRP), P-glycoprotein (P-gp, also called “ABCB1”) and breast cancer resistance protein (BCRP, also called “ABCG2”) ABC transporters. Among them, the expression of BCRP is the highest. Among these ABC transporters, the structures of MDR1, MRP1 and ABCG2 are shown in FIG. 1.

Substrates of such ABC transporters include various antitumor drugs such as cimetidine, toxins such as pheophorbide-a and PhIP, endogenous substrates such as estrone 3-sulfate, folic acid, estradial sulfate, 17-estradial-17-(-D-gluconide) and protoporphyrin IX(PPX), and fluorescent dyes such as Hoechest 33342, BODIPY-prazosin, and BBR 3390.

Among such ABC transporter substrates, antitumor drugs or toxins such as pheophorbide-a and PhIP may be used, and in one embodiment of the present invention, Mitoxantrone (MX), an antitumor drug, was used.

The antitumor drug, used in the present invention, is preferably a substrate of ABC transporters of the ABCG2 family, and in some cases, the antitumor drug is preferably a common substrate of ABCG2-family ABC transporters and MDR1-family ABC transporters, including MX, TOPOT (topotecan) or BISNAT (bisanthrone).

The toxin, used in the present invention, is preferably pheophorbide-a or 2-amino-1-methyl-6-phenylimidazo[4,5-b]pyridine (PHIP).

The step of bringing the substrate into contact with the stem cell culture to allow the substrate to react with the stem cell culture preferably comprises a step in which the substrate introduced into the cultured cells is released with the ABC transporters contained in cells to the outside of the ABC transporter-containing cells.

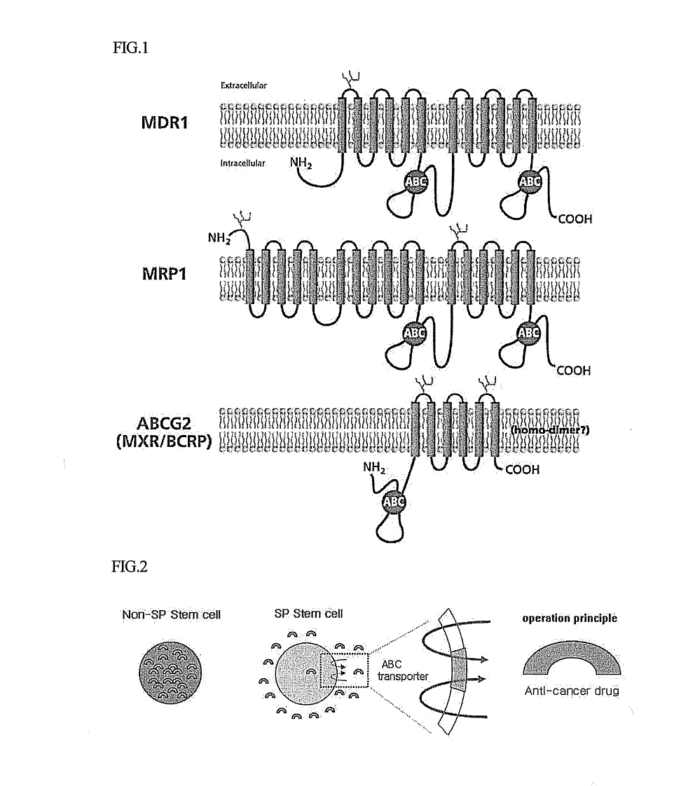

When the culture of stem cells is conducted, the stem cells are cultured together with differentiated cells, and thus the stem cell culture contains a mixture of the stem cells and the differentiated cells. In order to separate only the differentiated cells from the mixture of the stem cells and the differentiated cells, the stem cell culture is brought into contact with the antitumor drug or toxin ABC transporter substrate, as shown in FIG. 2, considering the fact that only the stem cells contain the ABC transporters. In this case, the substrate is introduced into the stem cells and the differentiated cells, and the substrate thus introduced into the cells is released to the outside of the stem cells due to the ABC transporters, but is not released from the differentiated cells.

Thus, the substrate antitumor or toxin introduced into the differentiated cells exists in the differentiated cells without being released from the cells, to kill the differentiated cells. On the other hand, the substrate introduced into the stem cells is released from the stem cells, such that the stem cells do not substantially contain the stem cells. Thus, the stem cells are not influenced by the substrate antitumor drug or toxin so as to survive. Accordingly, the killed differentiated cells and the viable stem cells can be separated from each other.

To kill the differentiated cells while making only the stem cells viable, the ABC transporter substrate as an antitumor drug or toxin is preferably used in a concentration of 0.2-3 M, more preferably 0.2-1 M and most preferably 0.2-0.4 M, per 10⁴ cultured cells.

The present invention may comprise a step of separating only stem cells having no substrate introduced therein, from the stem cell culture which reacted with the substrate. The cell separation in this step is preferably performed using the characteristics of the differentiated cells having the substrate introduced therein and the stem cells having substrate introduced therein. Specifically, the differentiated cells having the substrate introduced therein will be killed by the antitumor drug or toxin substrate, but the stem cells having no substrate introduced therein will be viable, because the substrate will have no effect on the activity of the stem cells. Accordingly, the killed differentiated cells and the viable stem cells can be separated from each other using a difference in characteristics between the killed cells and the viable cells, i.e., a difference in cell activity. Regarding methods of performing cell separation using this difference in cell activity, if stem cells have a characteristic in that these grow adhered to surfaces, killed differentiated cells will float, and viable stem cells will still be adhered to surfaces. In this case, a method of easily separating only the stem cells by removing the floating fluid can thus be used. Alternatively, a method of performing cell separation using the difference in dielectric property between the killed differentiated cells and the viable stem cells can be used.

Hereinafter, the present invention will be described in further detail with reference to examples. It to be understood, however, that these examples are for illustrative purposes only and are not to be construed to limit the scope of the present invention.

### EXAMPLE 1

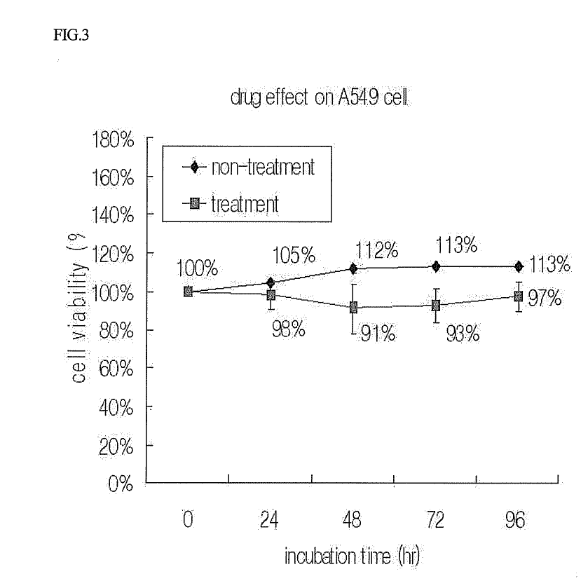

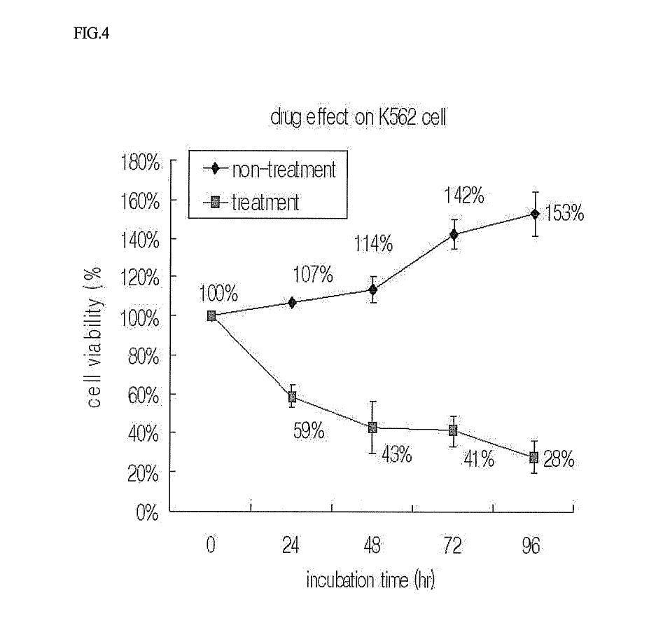

2×10⁴ cells of A549 (human lug carcinoma cell ATCC no.CCL-185), having ABC transporters of the ABCG2 family, were dispensed into each well and treated with Mitoxantrone (dissolved in PBS) at a Mitoxantrone concentration of 0.4 M. Then, the cells were incubated for 96 hours, while the cell activity was measured using an in vitro toxicology kit (Sulforhodamine B, Sigma) at 24-hour intervals. The measurement results are shown in FIG. 3. The results in FIG. 3 are expressed as the average of three measurements.

### COMPARATIVE EXAMPLE 1

This was carried out in the same manner as in Example 1, except that the cells were not treated with Mitoxantrone. The measurement results are shown in Table 3.

### EXAMPLE 2

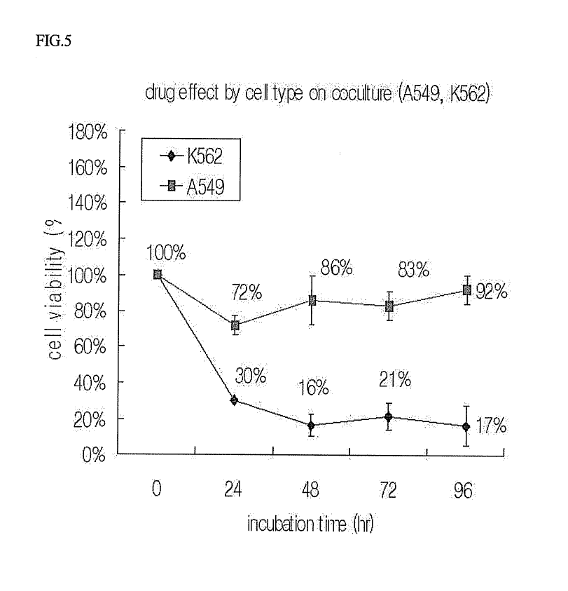

This was performed in the same manner as in Example 1, except that K562 (chronic myelogenous leukemia) cells having no ABC transporter were incubated in place of the A549 cells. The measurement results are shown in FIG. 4.

### COMPARATIVE EXAMPLE 2

This was carried out in the same manner as in Example 2, except that the cells were not treated with Mitoxantrone. The measurement results are shown in Table 4.

### EXAMPLE 3

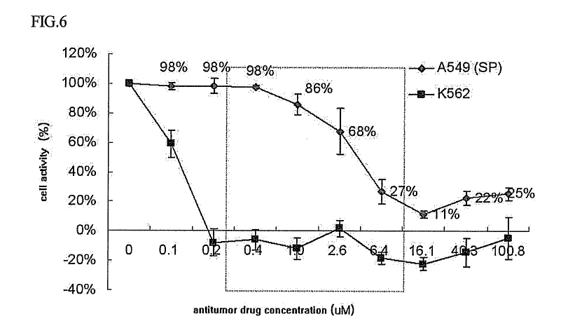

This was performed in the same manner as in Example 1, except that 10⁴ cells of A549, having ABC transporters of the ABCG2 family, and 10⁴ cells of K562, having no ABC transporter, were dispensed into each well and cocultured. The measurement results are shown in FIG. 5.

As shown in FIGS. 3 to 5, when the A549 cells having ABC transporters were incubated for 96 hours without treatment with Mitoxantrone, which is a substrate of ABC transporters of the ABCG2 family, the viability thereof was 113%. However, when the A549 cells were treated with Mitoxantrone and then incubated for 96 hours, the viability thereof was 97%, and when they were co-cultured with the K562 cells, the viability thereof was 92%. This suggests that there is no great difference in cell viability between the single culture of the cells treated with Mitoxantrone and the co-culture of the cells with other cells.

Meanwhile, when the K562 cells having no ABC transporter were incubated for 96 hours without treatment with antitumor Mitoxantrone, which is a substrate of ABC transporters of the ABCG2 family, the viability thereof was 153%. However, when the K562 cells were treated with Mitoxantrone and incubated for 96 hours, the viability thereof was 28% for single culture and 17% for coculture with the A549 cells. Thus, it can be seen that, when the K562 cells are co-cultured with cells having ABC transporters, the viability thereof is significantly decreased compared to when they are incubated alone after treatment with Mitoxantrone. The reason for this is believed to be as follows. When the K562 cells having no ABC transporters are co-cultured with A549 cells having ABC transporters, the A549 cells will introduce Mitoxantrone and then release the Mitoxantrone, while the local drug concentration around the K562 cells will increase, so that the K562 cells can be killed faster.

### TEST EXAMPLE 1

To examine cell activity as a function of the concentration of Mitoxantrone, 2×10⁴ cells of A549, having ABC transporters of the ABCG2 family, were dispensed into each well of a 96-well plate and treated with Mitoxantrone (dissolved in PBS) at Mitoxantrone concentrations of 0.1M, 0.2M, 0.4M, 1.0M, 2.6M, 6.4M, 16.1M, 40.3M and 100.8M. Then, the 96-well plate treated as described above was incubated for 96 hours, while the cell activity was measured using an in vitro toxicology kit (Sulforhodamine B, Sigma) at 24-hour intervals. The measurement results are shown in FIG. 6. The results in FIG. 6 are expressed as the average of three measurements.

### TEST EXAMPLE 2

This was performed in the same manner as in Example 1, except that K562 cells having no ABC transporter were used in place of the A549 cells. The measurement results are shown in FIG. 6.

As can be seen in FIG. 6, the A549 cells having ABC transporters had a cell activity of 98% at a Mitoxantrone concentration of 0.4M, a cell activity of 86% at a Mitoxantrone concentration of 1.0M, and a cell activity of 68% at a Mitoxantrone concentration of 2.6. Also, the K562 cells having no ABC transporter had a cell activity of about 60% at a Mitoxantrone concentration of 0.1M and a cell activity of almost zero (little or no viable cell) at a Mitoxantrone concentration of 0.2M.

Thus, it can be seen that the concentration of the antitumor or toxin ABC transporter substrate for killing cells having no ABC transporter while causing cells having ABC transporters to have activity is preferably 0.2-3M, more preferably 0.2-1M, and most preferably 0.2-0.4M, per 10⁴ cultured cells.

In the above Examples and Test Examples, the culture condition of stem cells was designed by mixing the A549 cell having ABC transporters with the K562 cells having no ABC transporters, without using the stem cell culture intact considering the fact that, in the culture process of the stem cells, differentiated cells have no ABC transporters, but the stem cells have ABC transporters. Thus, it is expected that, when the results of Examples and Test Examples are applied to a stem cell culture, the results of the stem cell culture will not be greatly different from the results of the Examples and Test Examples.

As described above, when the stem cell culture is brought into contact with the antitumor drug or toxin ABC transporter substrate such as Mitoxantrone so as to allow the substrate to react with the stem cell culture, the Mitoxantrone is introduced into the culture cells. At this time, differentiated cells having no ABC transporter retains the introduced Mitoxantrone without releasing the Mitoxantrone to the outside thereof, and thus are killed, and the stem cells having ABC transporters release the Mitoxantrone introduced therein to the outside thereof through the ABC transporters, so that the Mitoxantrone does not substantially exist in the stem cells, and thus has little or no effect on the activity of the stem cells.

Accordingly, the differentiated cells having Mitoxantrone introduced therein, and the stem cells from which Mitoxantrone was released to the outside thereof, can be separated from each other using either the characteristics of the cells themselves or FACS or MACS, and the purity of the stem cell culture can be increased by culturing only the separated cells from which the substrate was released.

As described above, the method of culturing stem cells using ABC transporters according to the present invention has an excellent effect capable of increasing the purity of cultured stem cells by selectively removing only differentiated cells having no ABC transporter from a stem cell culture comprising stem cells, containing a plurality of ABC transporters, and the differentiated cells, using an antitumor drug or toxin ABC transporter substrate.

Although the preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A method of culturing stem cells using ABC transporters, the method comprising the steps of:
bringing a stem cell culture into contact with an antitumor drug or toxin as a substrate of ATP-Binding Cassette transporters (ABC transporters) to allow the substrate to react with the stem cell culture; and
reculturing viable cells among the stem cell culture which reacted with the substrate, the viable cells having no substrate introduced therein.

2. The method of claim 1, wherein the antitumor drug is a substrate of ABC transporters of the ABCG2 family.

3. The method of claim 1, wherein the antitumor drug is a common substrate of ABCG2-family ABC transporter and MDR1-family ABC transporters.

4. The method of claim 3, wherein the antitumor drug is mitoxantrone (MX).

5. The method of claim 1, wherein the toxin is pheophorbide-a or 2-amino-1-methyl-6-phenylimidazo[4,5-b]pyridine (PhIP).

6. The method of claim 1, wherein the substrate is brought into contact with a concentration of 0.2-3M per 10⁴ cultured cells.

7. The method of claim 1, wherein the step of bringing the stem cell culture into contact with the substrate to allow the stem cell culture to contact with the substrate comprises a step in which the substrate introduced into the cultured cells is released with the ABC transporters contained in the stem cell to the outside of the stem cells.

8. The method of claim 1, wherein the step of reculturing the viable cells is carried out without a process of separating stem cells having no substrate introduced therein, from differentiated cells having the substrate introduced therein.

9. The method of claim 1, wherein the step of reculturing the viable cells is carried out after separating only stem cells having no substrate introduced therein, from differentiated cells having the substrate introduced therein.

10. The method of claim 9, wherein the cell separation is carried out using DEP, FACS, or MACS.

11. The method of claim 2, wherein the substrate is brought into contact with a concentration of 0.2-3M per 10⁴ cultured cells.

12. The method of claim 3, wherein the substrate is brought into contact with a concentration of 0.2-3M per 10⁴ cultured cells.

13. The method of claim 4, wherein the substrate is brought into contact with a concentration of 0.2-3M per 10⁴ cultured cells.

14. The method of claim 5, wherein the substrate is brought into contact with a concentration of 0.2-3M per 10⁴ cultured cells.
